(19) United States
(12) Patent Application Publication
(10) Pub. No.: US 2026/0115434 A1
(43) Pub. Date: Apr. 30, 2026

(54) DENTAL ORAL DRAINAGE TUBE

(71) Applicant: Cheol Hee JEONG, Seoul (KR)

(72) Inventor: Cheol Hee JEONG, Seoul (KR)

(21) Appl. No.: 19/310,954

(22) Filed: Aug. 27, 2025

(30) Foreign Application Priority Data
Oct. 28, 2024 (KR) 10-2024-0148192

(51) Int. Cl. A61M 27/00

(52) U.S. Cl. CPC A61M 27/00; A61M 2207/10

(57) ABSTRACT

The present invention relates to a dental oral drainage tube which, in dentistry or oral and maxillofacial surgery, is inserted into an infection site, such as an abscess or cellulitis, or into a site where pain and edema are expected after surgery, to drain pus, tissue fluid, or blood to the outside and prevent pooling around the surgical site. The drainage tube extends in a longitudinal direction, includes a radiopaque line on an outer or inner surface, and has a body providing a passage for fluid movement. A drainage hole is formed in the longitudinal direction of the body to introduce pus, tissue fluid, or blood into the passage, and a plurality of notch lines are formed on an outer surface of the body to limit the moving direction, thereby minimizing discomfort, preventing separation from the insertion position, and shortening the recovery period after surgery.

### CROSS-REFERENCE TO RELATED APPLICATION

This application claims priority to and the benefit of Korean Patent Application No. 10-2024-0148192, filed on Oct. 28, 2004, the disclosure of which is incorporated herein by reference in its entirety.

### BACKGROUND

### Field of the Invention

The present invention relates to a dental oral drainage tube, and more particularly, to a dental oral drainage tube provided as a drainage tube inserted into an infection site where pus is formed, such as an abscess and cellulitis, and a site where severe edema is expected after surgery in order to drain pus and infected blood to the outside and prevent the pus and infected blood from pooling in dentistry or oral and maxillofacial surgery, the dental oral drainage tube being capable of minimizing discomfort and shortening a recovery period after surgery by introducing pus, tissue fluid, or blood outside the drainage tube into a passage formed in the drainage tube to drain the pus, tissue fluid, or blood and being prevented from arbitrarily separating or falling from an insertion position.

### SUMMARY

### Discussion of Related Art

In dentistry or oral and maxillofacial surgery, a drainage tube made of a silicone material is inserted into a site where pain or edema is expected after surgery in order to allow blood or inflammation to be drained without pooling around the surgical site. To this end, most drainage tubes have the form of a tubular body that extends in a longitudinal direction and is hollow at the center and have a radiopaque line inserted in the form of a band into an outer surface or an inner surface in the longitudinal direction.

However, since the conventional drainage tubes are manufactured on the premise that they are inserted into a surgical site in the abdominal cavity instead of being manufactured exclusively for dentistry or oral and maxillofacial surgery, currently available drainage tubes generally have a cylindrical shape whose diameter is 6 mm and length is around 30 cm.

Therefore, there have been various problems in inserting drainage tubes manufactured for surgical sites in the abdominal cavity into oral cavity tissue having a property of being slippery due to relatively low viscosity and in which a surgical site is small or space for inserting a drainage tube is narrow as in dentistry or oral and maxillofacial surgery.

That is, due to a very large diameter of the existing drainage tubes, there have been an inconvenience of having to cut portions of the existing drainage tubes to form the drainage tubes into a plate shape and then insert the drainage tubes because it is not possible to insert the existing drainage tubes as it is into the corresponding site in a case in which surgery is performed on front teeth, canine teeth, premolar teeth, etc., excluding molar teeth.

In addition, the existing drainage tubes have problems that, due to having a slippery characteristic, it is not easy to insert them into a target site, it is difficult to maintain insertion positions thereof, and they easily fall out.

In addition, in the existing drainage tubes, a cylindrical tubular body extending in the longitudinal direction is formed into a plate shape, and because there is no component for allowing an outer surface and an inner surface to communicate, there is a problem that it is not possible to drain pus, tissue fluid, blood, or the like formed on the outer surface of the drainage tube. For example, in the case of an infection in the oral cavity (cellulitis, abscess), pus is formed throughout tissue at a side surface of the drainage tube instead of being formed below the drainage tube, and the existing drainage tubes are not able to drain pus, tissue fluid, or blood formed on the tissue at the side surface thereof.

This is because the existing drainage tubes are manufactured on the premise that they are inserted into a surgical site in the abdominal cavity as described above, and are manufactured on the premise that they need to drain only internal blood when inserted into a portion in the vicinity of a subcutaneous organ after surgery in the abdominal cavity (abdomen).

### BRIEF DESCRIPTION OF THE DRAWINGS

The above and other objects, features, and advantages of the present invention will become more apparent to those of ordinary skill in the art by describing exemplary embodiments thereof in detail with reference to the accompanying drawings, in which:

FIGS. 1(a) and 1(b) are exemplary views illustrating a dental oral drainage tube according to the present invention;

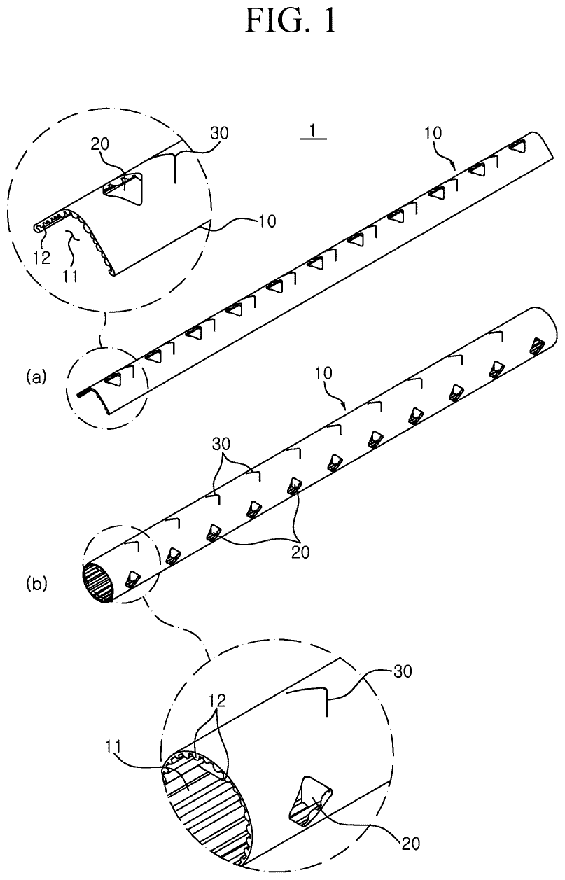

FIGS. 2(a) to 2(c) are exemplary views illustrating drainage holes according to different embodiments of the present invention;

FIG. 3 is an exemplary view illustrating notch lines constituting the present invention; and

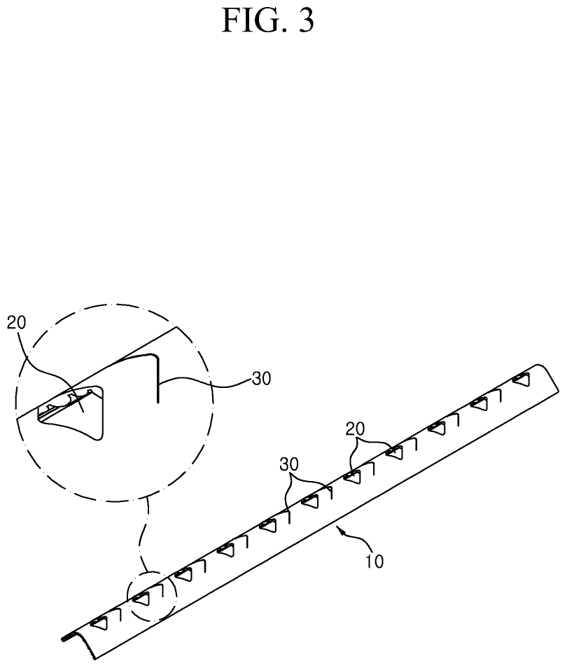

FIGS. 4(a) and 4(b) are exemplary views illustrating other embodiments of the notch lines constituting the present invention.

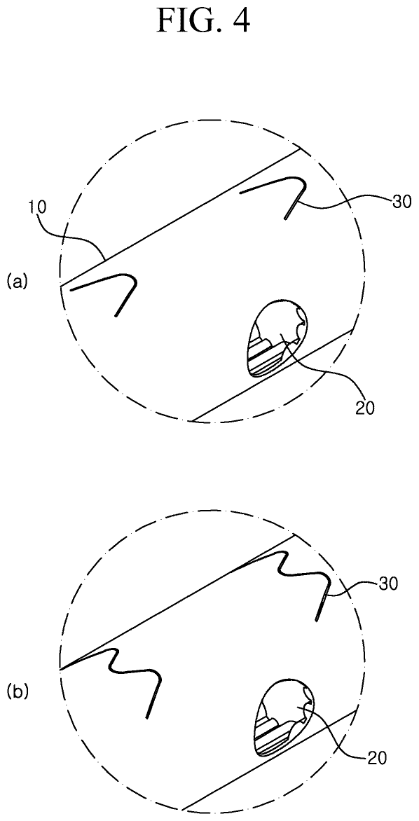

### DETAILED DESCRIPTION

Hereinafter, a preferred implementation example of a dental oral drainage tube according to the present invention will be described in detail with reference to the accompanying drawings.

First, a dental oral drainage tube 1 according to the present invention is a dental oral drainage tube that extends in a longitudinal direction, has a radiopaque line provided on any one of an outer surface and an inner surface, and is inserted into a site where pain and edema are expected after surgery to drain pus, tissue fluid, or blood to the outside and prevent the pus, tissue fluid, or blood from pooling around the surgical site, and the dental oral drainage tube 1 includes a body 10 extending in the longitudinal direction and providing a passage 11 along which the pus, tissue fluid, or blood moves, a drainage hole 20 formed in the longitudinal direction of the body 10 and introducing the pus, tissue fluid, or blood outside the body 10 into the passage, and a plurality of notch lines 30 formed on an outer surface of the body 10 to limit a moving direction of the body 10.

The body 10 is inserted into a surgical site to provide the passage 11 for draining pus, tissue fluid, or blood, the body 10 is formed to extend in the longitudinal direction, and the form and diameter of the body 10 may be changed according to a surgical site into which the dental oral drainage tube 1 is inserted.

For example, the body 10 inserted into a surgical site for premolar teeth may be formed in a plate shape having one side open because space of the surgical site into which the dental oral drainage tube 1 is inserted is narrow, and the body 10 inserted into a surgical site for molar teeth may be formed in the shape of a cylindrical tubular body having a predetermined diameter.

In addition, preferably, silicone may be used as a material forming the body 10.

In addition, a protrusion 12 protruding from the inner surface constituting the passage 11 toward the center of the passage 11 is formed, the protrusion 12 is provided as a plurality of protrusions 12 formed at predetermined intervals to guide a movement path of the pus, tissue fluid, or blood moving through the passage 11, and the protrusions 12 protrude to have heights different from each other.

In more detail, the passage 11 allows the pus, tissue fluid, or blood to move therethrough, and the pus, tissue fluid, or blood is a substance that has high viscosity and is slimy, and when the passage 11 is formed to be flat, there is a problem that a speed at which the pus, tissue fluid, or blood moves decreases on an inner surface of the passage 11, and thus a portion of the pus, tissue fluid, or blood congeals on the inner surface.

The protrusions 12 are formed to protrude from the inner surface forming the passage 11 toward the center of the passage 11, the protrusions 12 guide the pus, tissue fluid, or blood to move between a pair of protrusions 12 disposed adjacent to the passage 11 through which the pus, tissue fluid, or blood moves, and even when a portion of the moving pus, tissue fluid, or blood congeals between the pair of protrusions 12, if the portion congeals at a height lower than the heights of the protrusions 12, the movement speed of the pus, tissue fluid, or blood is not affected, and thus, drainage of the pus, tissue fluid, or blood can be smoothly performed.

In particular, since the protrusions 12 are formed to have heights different from each other and allow the pus, tissue fluid, or blood moving due to the protrusions 12 to vertically move due to the heights of the protrusions 12 while moving along the passage 11, stagnation of movement of the pus, tissue fluid, or blood is minimized, and congealing of the pus, tissue fluid, or blood is minimized.

The drainage hole 20 is provided as a plurality of drainage holes 20 formed at predetermined intervals from each other in the longitudinal direction of the body 10 to be penetrated from the outer surface of the body 10 toward the passage 11, and the drainage holes 20 allow tissue fluid, blood, pus, or the like formed at an outer side of the body 10 to be introduced into the passage 11 through the drainage holes 20 and drained.

The drainage holes may be formed in various shapes such as an elliptical shape, a polygonal shape including a quadrangular shape and a triangular shape, and other shapes including a start shape, a wave shape, etc., in addition to a circular shape illustrated in the drawings, and of course, the present invention is not limited to the embodiments illustrated in the drawings.

In addition, a diameter of the drainage holes 20 may be changed according to an outer diameter and a length of the body 10, an insertion site, and an expected amount of drainage, and of course, the present invention is not limited to proportions illustrated in the drawings.

In addition, in forming the plurality of drainage holes 20, of course, intervals therebetween may be changed according to the outer diameter and length of the body 10, the insertion site, and the expected amount of drainage.

In addition, the drainage holes 20 may be formed so that the diameter thereof gradually decreases or increases from the outer surface of the body 10 toward the inner surface thereof, and the drainage holes 20 may each be formed in the shape of a slot that extends in the longitudinal direction to correspond to the longitudinal direction of the body 10.

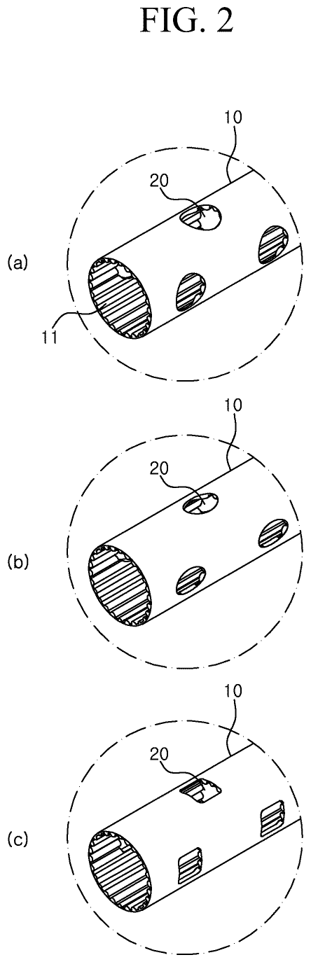

Further, as illustrated in FIGS. 2(a) to 2(c), the plurality of drainage holes 20 may be radially formed at predetermined intervals from each other on an outer circumference of the body 10, and here, the drainage holes 20 are formed at positions that do not intrude the radiopaque line provided on the body 10.

That is, for example, the drainage holes 20 may be formed at positions symmetrical to the radiopaque line, and based on the drainage holes 20 formed at the positions symmetrical to the radiopaque line, a pair of drainage holes 20 may be formed to be symmetrical to each other at positions rotated 90° to both sides along the outer surface of the body 10, and the pair of drainage holes 20 formed at the positions rotated 90° may be formed at positions relatively moved to one side in the longitudinal direction of the body 10 relative to the drainage holes 20 formed at the positions symmetrical to the radiopaque line.

The tissue fluid, blood, pus, or the like formed outside the body 10 is introduced into the passage 11 through the drainage holes 20 formed as described above and then is drained, and thus drainage efficiency can be increased.

In particular, a drainage hole according to one embodiment is provided as a plurality of drainage holes radially formed at predetermined intervals from each other on an outer circumference of the body, and on the body, a protrusion protruding from the inner surface constituting the passage toward the center of the passage 11 is formed, the protrusion is provided as a plurality of protrusions formed at predetermined intervals to guide a movement path of the pus, tissue fluid, or blood moving through the passage, and the protrusions may be formed to protrude to have heights different from each other.

The drainage holes rapidly introduce pus, blood, or tissue fluid formed on all tissue planes in contact with the tubular body into the passage to minimize stagnation of body fluids. In particular, since uniform drainage is possible throughout portions around the tube even in a situation in which a separate cavity is not formed as in an infection in the oral cavity, a treatment period is shortened, and edema and pain are reduced compared to the existing in-out type drainage tube.

In addition, when a sterilized saline solution or a disinfecting fluid is injected through the plurality of drainage holes, since a plurality of openings formed between an outer wall of the tube and infected tissue promote dispersion of the washing fluid, efficiency of washing and irrigation is improved. Accordingly, the washing fluid uniformly reaches the entire surface of a lesion and effectively reduces a bacterial burden, and tissue irritation can be minimized even during repeated washing.

Of course, the positions and intervals at which the drainage holes 20 are formed may be changed as described above and are not limited to the embodiments described above.

The notch lines 30 are formed on the outer surface of the body 10 to limit the moving direction of the body 10, and for example, the notch lines 30 form resistance in a direction opposite to the direction in which the body 10 is inserted and prevent the body 10 from falling.

In more detail, first, the notch lines 30 are formed by cutting the outer surface of the body 10 and each have the shape of a diagonal triangle that gradually narrows in a movement blocking direction.

The notch lines each having the shape of a diagonal triangle generate slight reverse resistance even in a wet, slippery oral mucosa environment and suppress self-falling of the drainage tube. This allows the tube to be stably fixed even during daily activities of a patient, such as eating, speaking, and opening/closing the oral cavity, and thus there are effects of preventing the risk of ingesting or inhaling foreign matter due to falling of the tube and reducing a burden of re-insertion on medical staff.

Here, the notch lines 30 limit movement in a direction from both side ends of the diagonal triangle toward a vertex thereof, and when the body 10 is inserted into a surgical site, both side ends of the notch lines 30 are placed to enter the surgical site first, and then the body 10 is inserted.

By the vertices of the notch lines 30 being disposed toward a direction in which the body 10 falls, the inserted body 10 is prevented from being arbitrarily removed or from separating or falling from the position where the body 10 is inserted due to impact transmitted from the outside.

The notch lines 30 are formed by cutting the outer surface of the body 10 as described above, and when force is applied to arbitrarily separate the body 10, as the vertices of the notch lines 30 are lifted upward, the notch lines 30 come into contact with skin tissue, generate resistance, and obstruct movement of the body 10.

The body 10 may be allowed to fall to the outside by preventing resistance from being generated by the notch lines 30 with specialized work of an operator or use of a separate tool.

Here, the notch lines 30 are formed at predetermined intervals from each other on the outer surface of the body 10 and are preferably formed at positions where the drainage holes 20 described above are not formed. For example, the notch lines 30 may be formed on the radiopaque line provided on the body 10.

The radiopaque line is disposed in the form of a band that is continuous in the longitudinal direction of the body 10, and since the notch lines 30 are formed by cutting the outer surface of the body 10 into a predetermined form instead of forming pores, the notch lines 30 may generate resistance when required, without impeding the functions and effects of the radiopaque line.

The present invention configured as described above provides a drainage tube that can be inserted into a surgical site even in dentistry or oral and maxillofacial surgery, and since tissue fluid, blood, pus due to infection, or the like formed at an outer surface of the drainage tube can be drained, there are effects of minimizing discomfort and shortening a recovery period after surgery.

In addition, according to the present invention, since insertion into a surgical site is easy, a change in position and falling can be prevented in an inserted state, and thus convenience of insertion is improved, there are advantages that fatigue of an operator can be reduced, and discomfort of a patient can be minimized because there is no inconvenience of re-insertion due to falling.

Further, notch lines each having the shape of a diagonal triangle that are employed in the present invention generate slight reverse resistance even in a wet, slippery oral mucosa environment and significantly suppress self-falling of the drainage tube. This allows the tube to be stably fixed even during daily activities of a patient, such as eating, speaking, and opening/closing the oral cavity, and thus there are direct effects of preventing the risk of ingesting or inhaling foreign matter due to falling of the tube and reducing a burden of re-insertion on medical staff.

In addition, a plurality of drainage holes formed in an entire surface of a body rapidly introduce pus, blood, or tissue fluid formed on all tissue planes in contact with a tubular body into a passage to minimize stagnation of body fluids. In particular, since uniform drainage is possible throughout portions around the tube even in a situation in which a separate cavity is not formed as in an infection in the oral cavity, a treatment period is shortened, and edema and pain are significantly reduced compared to the existing in-out type drainage tube.

When a sterilized saline solution or a disinfecting fluid is injected through the same drainage hole, since a plurality of openings formed between an outer wall of the tube and infected tissue promote dispersion of the washing fluid, efficiency of washing and irrigation is improved. Accordingly, the washing fluid uniformly reaches the entire surface of a lesion and effectively reduces a bacterial burden, and tissue irritation can be minimized even during repeated washing.

The present invention has been described above using specific details such as specific components and using limited embodiments and drawings, which are only provided to assist in overall understanding of the present invention, and the present invention is not limited to the above embodiments, and those of ordinary skill in the art to which the present invention pertains may make various modifications and changes to the above description.

Therefore, the spirit of the present invention should not be defined as limited to the embodiments described herein, and anything within not only the scope of the claims below but also their equivalents or equivalent modifications should be construed as belonging to the scope of the spirit of the present invention.

What is claimed is:

1. A dental oral drainage tube that extends in a longitudinal direction, has a radiopaque line provided on any one of an outer surface and an inner surface, and is inserted into a site where pain and edema are expected after surgery to drain pus, tissue fluid, or blood to the outside and prevent the pus, tissue fluid, or blood from pooling around the surgical site, the dental oral drainage tube comprising:
a body (10) extending in the longitudinal direction and providing a passage (11) along which the pus, tissue fluid, or blood moves;
a drainage hole (20) formed in the longitudinal direction of the body (10) and introducing the pus, tissue fluid, or blood outside the body (10) into the passage; and
a plurality of notch lines (30) formed on an outer surface of the body (10) to limit a moving direction of the body (10),
wherein the notch lines (30) are formed by cutting the outer surface of the body (10) and each have the shape of a diagonal triangle that gradually narrows in a movement blocking direction,
the drainage hole (20) is provided as a plurality of drainage holes radially formed at predetermined intervals from each other on an outer circumference of the body (10),
on the body (10), a protrusion (12) protruding from the inner surface constituting the passage (11) toward the center of the passage (11) is formed, and
the protrusion (12) is provided as a plurality of protrusions formed at predetermined intervals to guide a movement path of the pus, tissue fluid, or blood moving through the passage (11), and the protrusions protrude to have heights different from each other.

2. The dental oral drainage tube of claim 1, wherein the body (10) is formed in any one of a plate shape and a circular shape to correspond to an insertion site.
